SEQUENCE LISTING (2 sequences)

SEQ ID NO: 1 - protein, 72 aa, Artificial
  Synthetic construct
RAAPLEEMEAPAADAIMSPEEELDGYEPEPLGKRPAVLPLLELVGESGNNTSTDGSLPSTPPPAEEEEDELY

SEQ ID NO: 2 - protein, 108 aa, Artificial
  Synthetic construct
VAIVKEGWLHKRGEYIKTWRPRYFLLKNDGTFIGYKERPQDVDQREAPLNNFSVAQCQLMKTERPRPNTFIIRCLQWTTVIERTFHVETPEEREEWTTAIQTVADGLK

(19) United States
(12) Patent Application Publication
(10) Pub. No.: US 2021/0137953 A1
(43) Pub. Date: May 13, 2021

(54) Inhibitors of Mcl-1 and Akt Binding, Pharmaceutical Compositions, and Uses in Treating Cancer

(71) Applicant: Emory University, Atlanta, GA (US)

(72) Inventor: Xingming Deng, Atlanta, GA (US)

(21) Appl. No.: 17/096,439

(22) Filed: Nov. 12, 2020

Related U.S. Application Data
(60) Provisional application No. 62/934,018, filed on Nov. 12, 2019.

(51) Int. Cl. A61K 31/675; A61P 35/00; A61K 45/06

(52) U.S. Cl. CPC A61K 31/675; A61K 45/06; A61P 35/00

(57) ABSTRACT

This disclosure relates to inhibitors of Mcl-1 and Akt binding, pharmaceutical compositions, and uses in treating cancer. In certain embodiments, this disclosure relates to methods of treating cancer comprising administering an effective amount of an inhibitor of Mcl-1 and Akt binding to a subject in need thereof. In certain embodiments, the inhibitor prevents the PEST domain of Mcl-1 from directly interacting with the pleckstrin homology (PH) domain of Akt.

### CROSS-REFERENCE TO RELATED APPLICATIONS

This application claims the benefit of U.S. Provisional Application No. 62/934,018 filed Nov. 12, 2019. The entirety of this application is hereby incorporated by reference for all purposes.

### STATEMENT REGARDING FEDERALLY SPONSORED RESEARCH OR DEVELOPMENT

This invention was made with government support under CA193828, CA136534, and CA200905 awarded by the National Institutes of Health. The government has certain rights in the invention.

### INCORPORATION-BY-REFERENCE OF MATERIAL SUBMITTED AS A TEXT FILE VIA THE OFFICE ELECTRONIC FILING SYSTEM (EFS-WEB)

The Sequence Listing associated with this application is provided in text format in lieu of a paper copy and is hereby incorporated by reference into the specification. The name of the text file containing the Sequence Listing is 20034US_ST25.txt. The text file is 3 KB, was created on Nov. 11, 2020, and is being submitted electronically via EFS-Web.

### BACKGROUND

Mcl-1 is a Bcl2 family member protein that plays a role in apoptosis and tumorigenesis. Structurally, Mcl-1 has a N-terminal end and lacks a typical BH4 domain compared with Bcl2, Bcl-xL, and Bcl-w. Mcl-1 encodes a long proline-, glutamic acid-, serine-, and threonine-rich (PEST) region upstream of the Bcl2 homology (BH) domain.

Zhao et al. report nicotine enhances the antiapoptotic function of Mcl-1 through phosphorylation. Mol Cancer Res, 2009,7:1954-61.

Song et al. report Mcl-1 regulates survival and sensitivity to diverse apoptotic stimuli in human non-small cell lung cancer cells. Cancer Biol Ther, 2005, 4:267-76.

Chen et al. report targeting Mcl-1 enhances DNA replication stress sensitivity to cancer therapy. J Clin Invest, 2018, 128(1): 500-516.

Akt functions as an oncogenic kinase that consists of an N terminal pleckstrin homology (PH) domain, a kinase domain (KD), and a C-terminal regulatory region carrying a hydrophobic motif. In response to growth factor stimulation, activation of PI3K produces phosphatidylinositol-3, 4, 5-bisphosphate (PIP3) that directly binds to the PH domain and induces a conformational change in Akt enabling PDK1 or mTORC2 to access and phosphorylate Akt at T308 within the catalytic domain or at S473 in the hydrophobic motif, respectively. Phosphorylation of T308 and S473 subsequently activates Akt and its downstream signaling.

Parikh et al. report the disruption of PH-kinase domain interactions leads to oncogenic activation of Akt in human cancers. Proc Natl Acad Sci USA, 2012, 109: 19368-73.

References cited herein are not an admission of prior art.

### SUMMARY

This disclosure relates to inhibitors of Mcl-1 and Akt binding, pharmaceutical compositions, and uses in treating cancer. In certain embodiments, this disclosure relates to methods of treating cancer comprising administering an effective amount of an inhibitor of Mcl-1 and Akt binding to a subject in need thereof. In certain embodiments, the inhibitor prevents the PEST domain of Mcl-1 from directly interacting with the pleckstrin homology (PH) domain of Akt.

In certain embodiments, the inhibitor is 2-(hydroxymethyl)-6-imino-2,3,3a,9a-tetrahydro-6H-furo[2′,3′:4,5]oxazolo[3,2-a]pyrimidin-3-yl dihydrogen phosphate (PH-687), derivative, ester, or salt thereof.

In certain embodiments, the inhibitor is a compound of formula I,

[chemical structure]

derivative, ester, or salt thereof, wherein substituents are reported herein. In certain embodiments, R¹ is alkyl substituted with hydroxy. In certain embodiments, R³ is hydroxy. In certain embodiments, R⁴ is hydroxy.

In certain embodiments, the subject is diagnosed with non-small cell lung cancer (NSCLC). In certain embodiments, the subject is diagnosed with a cancer selected from lung, pancreatic, colorectal, uterine, esophageal, gastric, cervical, breast, prostate, or bladder cancer.

In certain embodiments, the inhibitor of Mcl-1 and Akt binding is administered in combination with an additional chemotherapy agent.

In certain embodiments, this disclosure relates to pharmaceutical compositions comprising an inhibitor of Mcl-1 and Akt binding disclosed herein or pharmaceutically acceptable salt thereof and a pharmaceutically acceptable excipient.

In certain embodiments, the pharmaceutical is in the form of a pill, capsule, or table.

In certain embodiments, the pharmaceutical composition is in the form of an aqueous isotonic or non-isotonic pH buffered solution.

In certain embodiments, this disclosure relates to methods of diagnosing and treating a subject comprising measuring levels of Mcl-1 and/or measuring levels of Akt from a sample of the subject; comparing the measured levels to Mcl-1 and/or Akt to a reference or normal value; wherein if the measured levels are higher than the reference or normal values, administering an effective amount of an inhibitor of Mcl-1 and Akt binding, alternative chemotherapy treatment, a combination chemotherapy treatment, or an aggressive chemotherapy treatment to the subject.

In certain embodiments, the sample is a tumor or tissue sample.

In certain embodiments, the subject is diagnosed with lung cancer, e.g., NSCLC.

### BRIEF DESCRIPTION OF THE SEVERAL VIEWS OF THE DRAWINGS

FIG. 1 shows data indicating that knockout of Mcl-1 inhibits tumor growth in vivo. The same number (3×10⁶) of H1299 parental or Mcl-1⁻/⁻ cells were injected into subcutaneous tissue in the flank region of nude mice to generate lung cancer xenografts (n ¼ 5 mice each group). Tumor volume was measured once every 3 days. After 33 days, the mice were sacrificed and the tumors were removed, photographed, and analyzed.

FIG. 2 shows data where H1299 Mcl-1⁻/⁻ cells expressing empty vector, Flag-tagged WT Mcl-1 or DPEST mutant were injected into subcutaneous tissue in the flank region of nude mice to generate lung cancer xenografts. Tumor volume was measured once every 3 days. After 33 days, the mice were sacrificed and the tumors were removed, photographed, and analyzed.

FIG. 3 illustrates the chemical structure of PH-687 and shows data from colony formation assays using H1299, H157, and H460 cells following treatment with increasing concentrations of PH-687.

FIG. 4 shows data indicating PH-687 suppresses tumor growth in vivo. Nu/Nu nude mice with H1299 lung cancer xenografts were treated with increasing doses of PH-687 for 4 weeks. Tumor volume was measured once every 4 days. After 28 days, mice were sacrificed and the tumors were removed, photographed, and analyzed.

### DETAILED DESCRIPTION

Before the present disclosure is described in greater detail, it is to be understood that this disclosure is not limited to embodiments described, and as such may, of course, vary. It is also to be understood that the terminology used herein is for the purpose of describing particular embodiments only and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

To the extent that any chemical formulas reported herein contain one or more chiral centers, the formulas are intended to encompass all stable stereoisomers, enantiomers, and diastereomers. It is also understood that formulas encompass all tautomeric forms.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent. “Mcl-1” refers to induced myeloid leukemia cell differentiation protein Mcl-1. During gene expression, alternative splicing results in multiple transcript variants. The longest gene product (isoform 1) is reported to enhance cell survival by inhibiting apoptosis while the alternatively spliced shorter gene products (isoform 2 and isoform 3) are reported to promote apoptosis. Isoforms 1 and 2 contain the PEST domain identified as amino acids 104-175 having the following sequence RAAPLEE MEAPAADAIM SPEEELDGYE PEPLGKRPAV LPLLELVGES GNNTSTDGSL PSTPPPAEEE EDELY (SEQ ID NO: 1). Human isoform 1 [Homo sapiens] is denoted by NCBI Reference Sequence: NP_068779.1. Human isoform 2 is denoted as NCBI Reference Sequence: NP_877495.1.

“Akt” refers to the serine/threonine-protein kinases (e.g., AKT1, AKT2 and AKT3) which regulate many processes including metabolism, proliferation, cell survival, growth and angiogenesis. Protein product of AKT1 gene is also known as RAC-alpha serine/threonine-protein kinase. Akt proteins have an N-terminal pleckstrin homology domain, a serine/threonine-specific kinase domain and a C-terminal regulatory domain. These proteins are phosphorylated by phosphoinositide 3-kinase (PI3K). Human AKT1 has NCBI Reference Sequence: NP_001014431.1. Protein Kinase B-like pleckstrin homology (PH) domain is identified as amino acids 4-111 with the sequence VAIVKEG WLHKRGEYIK TWRPRYFLLK NDGTFIGYKE RPQDVDQREA PLNNFSVAQC QLMKTERPRP NTFIIRCLQW TTVIERTFHV ETPEEREEWT TAIQTVADGL K (SEQ ID NO: 2).

As used herein, “subject” refers any animal, preferably a human patient, livestock, or domestic pet.

As used herein, the terms “treat” and “treating” are not limited to the case where the subject (e.g. patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

As used herein, “salts” refer to derivatives of the disclosed compounds where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like. In certain embodiments, the salts are conventional nontoxic pharmaceutically acceptable salts including the quaternary ammonium salts of the parent compound formed, and non-toxic inorganic or organic acids.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing an oxygen atom with a sulphur atom or replacing an amino group with a hydroxyl group. The derivative may be a prodrug. Derivatives may be prepared by any variety of synthetic methods or appropriate adaptations presented in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRₐR_{b}, —NRₐC(═O)R_{b}, —NRₐC(═O)NRₐNR_{b}, —NRₐC(═O) OR_{b}, —NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ and —S(═O)₂ORₐ. Rₐ and R_{b} in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, or heteroarylalkyl.

As used herein, the term “prodrug” refers a compound that, after administration, is metabolized (i.e., converted within the body) into a pharmacologically active drug. Examples include alkoxy esters of hydroxyl groups or carboxyl groups such as acetate esters, benzoate esters, alkyl ethers, amino acids esters, glycolic acid esters, malic acid esters, acyloxyalkyl esters, alkoxycarbonyloxy alkyl esters, S-acylthioalkyl esters, hydroxylamine amides, phosphonylmethoxy ethers, phosphates, phosphoramidates, and combinations thereof.

The prodrug may also have improved solubility in pharmaceutical compositions over the parent drug. A prodrug may be converted into the parent drug by various mechanisms, including enzymatic processes and metabolic hydrolysis. Typical prodrugs are pharmaceutically acceptable esters. Prodrugs include compounds wherein a hydroxy, amino or mercapto group is bonded to any group that, when the prodrug of the active compound is administered to a subject, cleaves to form a free hydroxy, free amino or free mercapto group, respectively.

If a disclosed compound or a pharmaceutically acceptable form of the compound contains an alcohol functional group, a prodrug can be formed by the replacement of the hydrogen atom of the alcohol group with a group such as (C₁-C₆)(alkanoyloxy)methyl, 1-((C₁-C₆)alkanoyloxy) ethyl, 1-methyl-1((C₁-C₆)alkanoyloxy)ethyl (C₁-C₆)(alkoxycarbonyloxy)methyl, N-(C₁-C₆)alkoxycarbonylaminomethyl, succinoyl, (C₁-C₆)alkanoyl, alpha-amino(C₁-C₄)alkanoyl, arylacyl and alpha-aminoacyl, or alpha-aminoacyl-alpha-aminoacyl, where each alpha-aminoacyl group is independently selected from naturally occurring L-amino acids —P(O)(OH)₂, —P(O)(O(C₁-C₆)alkyl)₂, and glycosyl (the radical resulting from the removal of a hydroxyl group of the hemiacetal form of a carbohydrate).

If a disclosed compound or a pharmaceutically acceptable form of the compound incorporates an amine functional group, a prodrug can be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, a natural alpha-aminoacyl, —C(OH)C(O)OY₁ wherein Y¹ is H, (C₁-C₆)alkyl or benzyl, —C(OY₂)Y₃ wherein Y₂ is (C₁-C₄) alkyl and Y₃ is (C₁-C₆)alkyl, carboxy(C₁-C₆)alkyl, amino(C₁-C₄)alkyl or mono-Nor di-N,N—(C₁-C₆)alkylaminoalkyl, —C(Y₄)Y₅ wherein Y₄ is H or methyl and Y₅ is mono-N— or di-N,N—(C₁-C₆)alkylamino, morpholino, piperidin-1-yl or pyrrolidin-1-yl.

As used herein, “alkyl” means a noncyclic straight chain or branched, unsaturated or saturated hydrocarbon such as those containing from 1 to 25 carbon atoms. For example, a “C₈-C₁₈” refers to an alkyl containing 8 to 18 carbon atoms. Likewise, a “C₆-C₂₂” refers to an alkyl containing 6 to 22 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

Non-aromatic mono or polycyclic alkyls are referred to herein as “carbocycles” or “carbocyclyl” groups. Representative saturated carbocycles include cyclopropyl, cyclobutyl, cyclopentyl, cyclohexyl, and the like; while unsaturated carbocycles include cyclopentenyl and cyclohexenyl, and the like.

“Heterocarbocycles” or heterocarbocyclyl” groups are carbocycles which contain from 1 to 4 heteroatoms independently selected from nitrogen, oxygen and sulphur which may be saturated or unsaturated (but not aromatic), monocyclic or polycyclic, and wherein the nitrogen and sulphur heteroatoms may be optionally oxidized, and the nitrogen heteroatom may be optionally quaternized. Heterocarbocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, and the like.

The term “aryl” refers to aromatic homocyclic (i.e., hydrocarbon) mono-, bi- or tricyclic ring-containing groups preferably having 6 to 12 members such as phenyl, naphthyl and biphenyl. Phenyl is a preferred aryl group.

As used herein, “heteroaryl” or “heteroaromatic” refers an aromatic heterocarbocycle having 1 to 4 heteroatoms selected from nitrogen, oxygen and sulfur, and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl sub stituent.

As used herein, “heterocycle” or “heterocyclyl” refers to mono- and polycyclic ring systems having 1 to 4 heteroatoms selected from nitrogen, oxygen and sulfur, and containing at least 1 carbon atom. The mono- and polycyclic ring systems may be aromatic, non-aromatic or mixtures of aromatic and non-aromatic rings. Heterocycle includes heterocarbocycles, heteroaryls, and the like.

“Alkoxy” refers to an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, t-butoxy.

“Alkoxyalkyl” refers an alkyl group as defined above with the indicated number of carbon atoms attached through an alkyl bridge (i.e., —CH₂—O—CH₂CH₃).

“Alkylamino” refers an alkyl group as defined above with the indicated number of carbon atoms attached through an amino bridge. An example of an alkylamino is methylamino, (i.e., —NH—CH₃).

“Alkylthio” refers to an alkyl group as defined above with the indicated number of carbon atoms attached through a sulfur bridge. An example of an alkylthio is methylthio, (i.e., —S—CH₃).

“Alkanoyl” refers to an alkyl as defined above with the indicated number of carbon atoms attached through a carbonyl bride (i.e., —(C═O)alkyl).

The terms “cycloalkyl” and “cycloalkenyl” refer to mono-, bi-, or tri homocyclic ring groups of 3 to 15 carbon atoms which are, respectively, fully saturated and partially unsaturated.

“Alkylsulfonyl” refers to an alkyl as defined above with the indicated number of carbon atoms attached through a sulfonyl bridge (i.e., —S(═O)₂alkyl) such as mesyl and the like, and “Arylsulfonyl” refers to an aryl attached through a sulfonyl bridge (i.e., —S(═O)₂aryl).

“Alkylsulfamoyl” refers to an alkyl as defined above with the indicated number of carbon atoms attached through a sulfamoyl bridge (i.e., —NHS(═O)₂alkyl), and an “Arylsulfamoyl” refers to an alkyl attached through a sulfamoyl bridge (i.e., (i.e., —NHS(═O)₂aryl).

“Alkylsulfinyl” refers to an alkyl as defined above with the indicated number of carbon atoms attached through a sulfinyl bridge (i.e. —S(═O)alkyl).

The terms “halogen” and “halo” refer to fluorine, chlorine, bromine, and iodine.

A “linking group” refers to any variety of molecular arrangements that can be used to bridge to molecular moieties together. An example formula may be —Rₙ— wherein R is selected individually and independently at each occurrence as: —CRₙRₙ—, —CHRₙ—, —CH—, —C—, —CH₂—, —C(OH)Rₙ, —C(OH)(OH)—, —C(OH)H, —C(Hal)Rₙ—, —C(Hal)(Hal)—, —C(Hal)—, —C(N₃)Rₙ—, —C(CN)Rₙ—, —C(CN)(CN)—, —C(CN)H—, —C(N₃)(N₃)—, —C(N₃)H—, —O—, —S—, —N—, —NH—, —NRₙ—, —(C═O)—, —(C═NH)—, —(C═S)—, —(C═CH₂)—, which may contain single, double, or triple bonds individually and independently between the R groups. If an R is branched with an Rₙ it may be terminated with a group such as —CH₃, —H, —CH═CH₂, —CCH, —OH, —SH, —NH₂, —N₃, —CN, or —Hal, or two branched Rs may form a cyclic structure. It is contemplated that in certain instances, the total Rs or “n” may be less than 100 or 50 or 25 or 10. Examples of linking groups include bridging alkyl groups and alkoxyalkyl groups.

In certain embodiments, this disclosure contemplates compound or composition as disclosed herein in the production of a medicament for use in treating cancer. “Cancer” refers any of various cellular diseases with malignant neoplasms characterized by the proliferation of cells. It is not intended that the diseased cells must actually invade surrounding tissue and metastasize to new body sites. Cancer can involve any tissue of the body and have many different forms in each body area. Within the context of certain embodiments, whether “cancer is reduced” may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample compound compared to a control without the compound. It may also be identified by a change in relevant biomarker or gene expression profile, such as PSA for prostate cancer, HER2 for breast cancer, or others.

The cancer to be treated in the context of the present disclosure may be any type of cancer or tumor such as lung cancer, non-small cell lung cancer and subtypes of NSCLC such as adenocarcinoma, squamous cell carcinoma, and large cell carcinoma, and small cell lung cancer. Contemplated are malignancies located in the colon, abdomen, bone, breast, digestive system, liver, pancreas, peritoneum, endocrine glands (adrenal, parathyroid, hypophysis, testicles, ovaries, thymus, thyroid), eye, head and neck, nervous system (central and peripheral), lymphatic system, pelvis, skin, soft tissue, spleen, thorax and genito-urinary apparatus and, more particularly, adrenocortical carcinoma, AIDS-related lymphoma, AIDS-related malignant tumors, anal cancer, astrocytoma, cancer of the biliary tract, cancer of the bladder, bone cancer, brain stem glioma, brain tumors, breast cancer, cancer of the renal pelvis and ureter, primary central nervous system cerebellar astrocytoma, brain astrocytoma, cancer of the cervix, chronic lymphocytic leukemia, chronic myeloid leukemia, cancer of the colon, cutaneous T-cell lymphoma, endocrine pancreatic islet cells carcinoma, endometrial cancer, ependymoma, epithelial cancer, cancer of the esophagus, Ewing's sarcoma and related tumors, cancer of the exocrine pancreas, extracranial germ cell tumor, extragonadal germ cell tumor, extrahepatic biliary tract cancer, cancer of the eye, Gaucher's disease, cancer of the gallbladder, gastric cancer, gastrointestinal carcinoid tumor, gastrointestinal tumors, germ cell tumors, gestational trophoblastic tumor, head and neck cancer, hepatocellular cancer, hypergammaglobulinemia, hypopharyngeal cancer, Hodgkin's disease, intestinal cancers, intraocular melanoma, islet cell carcinoma, islet cell pancreatic cancer, Kaposi's sarcoma, cancer of the larynx, cancer of the lip and mouth, macroglobulinemia, malignant mesothelioma, malignant thymoma, medulloblastoma, melanoma, mesothelioma, occult primary metastatic squamous neck cancer, primary metastatic squamous neck cancer, metastatic squamous neck cancer, multiple myeloma, multiple myeloma/plasmatic cell neoplasia, myelodysplastic syndrome, myelogenous leukemia, myeloid leukemia, myeloproliferative disorders, paranasal sinus and nasal cavity cancer, nasopharyngeal cancer, neuroblastoma, non-Hodgkin's lymphoma, non-melanoma skin cancer, non-small cell lung cancer, metastatic squamous neck cancer with occult primary, buccopharyngeal cancer, malignant fibrous histiocytoma, malignant fibrous osteosarcoma/histiocytoma of the bone, epithelial ovarian cancer, ovarian germ cell tumor, ovarian low malignant potential tumor, pancreatic cancer, paraproteinemias, purpura, parathyroid cancer, cancer of the penis, hypophysis tumor, neoplasia of plasmatic cells/multiple myeloma, primary central nervous system lymphoma, primary liver cancer, prostate cancer, rectal cancer, renal cell cancer, cancer of the renal pelvis and ureter, retinoblastoma, rhabdomyosarcoma, cancer of the salivary glands, sarcoidosis, sarcomas, skin cancer, small cell lung cancer, small intestine cancer, soft tissue sarcoma, squamous neck cancer, stomach cancer, pineal and supratentorial primitive neuroectodermal tumors, T-cell lymphoma, testicular cancer, thymoma, thyroid cancer, transitional cell cancer of the renal pelvis and ureter, transitional renal pelvis and ureter cancer, trophoblastic tumors, cell cancer of the renal pelvis and ureter, cancer of the urethra, cancer of the uterus, uterine sarcoma, vaginal cancer, optic pathway and hypothalamic glioma, cancer of the vulva, Waldenstrom's macroglobulinemia, Wilms' tumor and any other hyperproliferative disease, as well as neoplasia, located in the system of a previously mentioned organ.

In certain embodiments, compounds disclosed herein may be administered in combination with an additional anti-cancer agent. A “chemotherapy agent,” “chemotherapeutic,” “anti-cancer agent” or the like, refer to molecules that are recognized to aid in the treatment of a cancer. Contemplated examples include the following molecules or derivatives such as abemaciclib, abiraterone acetate, methotrexate, paclitaxel, adriamycin, acalabrutinib, brentuximab vedotin, adotrastuzumab emtansine, aflibercept, afatinib, netupitant, palonosetron, imiquimod, aldesleukin, alectinib, alemtuzumab, pemetrexed disodium, copanlisib, melphalan, brigatinib, chlorambucil, amifostine, aminolevulinic acid, anastrozole, apalutamide, aprepitant, pamidronate disodium, exemestane, nelarabine, arsenic trioxide, ofatumumab, atezolizumab, bevacizumab, avelumab, axicabtagene ciloleucel, axitinib, azacitidine, carmustine, belinostat, bendamustine, inotuzumab ozogamicin, bevacizumab, bexarotene, bicalutamide, bleomycin, blinatumomab, bortezomib, bosutinib, brentuximab vedotin, brigatinib, busulfan, irinotecan, capecitabine, fluorouracil, carboplatin, carfilzomib, ceritinib, daunorubicin, cetuximab, cisplatin, cladribine, cyclophosphamide, clofarabine, cobimetinib, cabozantinib-S-malate, dactinomycin, crizotinib, ifosfamide, ramucirumab, cytarabine, dabrafenib, dacarbazine, decitabine, daratumumab, dasatinib, defibrotide, degarelix, denileukin diftitox, denosumab, dexamethasone, dexrazoxane, dinutuximab, docetaxel, doxorubicin, durvalumab, rasburicase, epirubicin, elotuzumab, oxaliplatin, eltrombopag olamine, enasidenib, enzalutamide, eribulin, vismodegib, erlotinib, etoposide, everolimus, raloxifene, toremifene, panobinostat, fulvestrant, letrozole, filgrastim, fludarabine, flutamide, pralatrexate, obinutuzumab, gefitinib, gemcitabine, gemtuzumab ozogamicin, glucarpidase, goserelin, propranolol, trastuzumab, topotecan, palbociclib, ibritumomab tiuxetan, ibrutinib, ponatinib, idarubicin, idelalisib, imatinib, talimogene laherparepvec, ipilimumab, romidepsin, ixabepilone, ixazomib, ruxolitinib, cabazitaxel, palifermin, pembrolizumab, rib ociclib, tisagenlecleucel, lanreotide, lapatinib, olaratumab, lenalidomide, lenvatinib, leucovorin, leuprolide, lomustine, trifluridine, olaparib, vincristine, procarbazine, mechlorethamine, megestrol, trametinib, temozolomide, methylnaltrexone bromide, midostaurin, mitomycin C, mitoxantrone, plerixafor, vinorelbine, necitumumab, neratinib, sorafenib, nilutamide, nilotinib, niraparib, nivolumab, tamoxifen, romiplostim, sonidegib, omacetaxine, pegaspargase, ondansetron, osimertinib, panitumumab, pazopanib, interferon alfa-2b, pertuzumab, pomalidomide, mercaptopurine, regorafenib, rituximab, rolapitant, rucaparib, siltuximab, sunitinib, thioguanine, temsirolimus, thalidomide, thiotepa, trabectedin, valrubicin, vandetanib, vinblastine, vemurafenib, vorinostat, zoledronic acid, or combinations thereof such as cyclophosphamide, methotrexate, 5-fluorouracil (CMF); doxorubicin, cyclophosphamide (AC); mustine, vincristine, procarbazine, prednisolone (MOPP); sdriamycin, bleomycin, vinblastine, dacarbazine (ABVD); cyclophosphamide, doxorubicin, vincristine, prednisolone (CHOP); rituximab, cyclophosphamide, doxorubicin, vincristine, prednisolone (RCHOP); bleomycin, etoposide, cisplatin (BEP); epirubicin, cisplatin, 5-fluorouracil (ECF); epirubicin, cisplatin, capecitabine (ECX); methotrexate, vincristine, doxorubicin, cisplatin (MVAC).

In certain embodiments, the chemotherapy agent is an anti-PD-1, anti-CTLA4 antibody or combinations thereof, such as an anti-CTLA4 (e.g., ipilimumab, tremelimumab) or an anti-PD1 antibody (e.g., nivolumab, pembrolizumab, atezolizumab, avelumab, durvalumab).

### Inhibitors of Mcl-1 and Akt Binding

Although it is not intended that certain embodiments, of this disclosure be limited by any particular mechanism, it is believed that certain compounds disclosed herein inhibit the PEST domain of Mcl-1 from directly interacting with the pleckstrin homology (PH) domain of Akt; thus, the compounds are useful therapeutic agents for treating cancer.

In certain embodiments, the inhibitor is 2-(hydroxymethyl)-6-imino-2,3,3a,9a-tetrahydro-6H-furo[2′,3′:4,5]oxazolo[3,2-a]pyrimidin-3-yl dihydrogen phosphate, derivative, ester, or salt thereof. In certain embodiments, the therapeutic agents can be defined as a compound of formula I,

[chemical structure]

derivatives, esters, or salts thereof, wherein:

Q is O or S;

U is a linking group, O, S, NH, or CH₂;

X is O, S, NH, or CH₂;

Y is O, S NH, or CH₂;

Z is N or CH;

R¹, R², R³, R⁴, R⁵, R⁶, R⁷, and R⁸, are each individually and independently hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, phosphate, alkylsulfinyl, alkyl sulfonyl, arylsulfonyl, carbocyclyl, aryl, or heterocyclyl, wherein R¹, R², R³, R⁴, R⁵, R⁶, R⁷ and R⁸ are optionally substituted with one or more, the same or different, R¹⁰ or R¹²;

R¹⁰ is alkyl, halogen, cyano, hydroxy, amino, mercapto, formyl, carboxy, carbamoyl, alkoxy, alkanoyl, alkylthio, alkylamino, phosphate, aminoalkyl, (alkyl)₂amino, alkylsulfinyl, alkylsulfonyl, arylsulfonyl, carbocyclyl, aryl, or heterocyclyl, wherein R¹⁰ is optionally substituted with one or more, the same or different, R¹¹ or R¹²;

R¹¹ is alkyl, halogen, cyano, hydroxy, amino, mercapto, formyl, carboxy, carbamoyl, alkoxy, alkanoyl, alkylthio, alkylamino, aminoalkyl, (alkyl)₂amino, phosphate, alkylsulfinyl, alkylsulfonyl, arylsulfonyl, carbocyclyl, aryl, or heterocyclyl, wherein R¹¹ is optionally substituted with one or more, the same or different, R¹²; and

R¹² is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, 2-methoxyethoxy, 2-hydroxyethoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, the R¹ is alkyl substituted with hydroxy. In certain embodiments, the R³ is hydroxy. In certain embodiments, the R⁴ is hydroxy.

In certain embodiments, it is contemplated that therapeutic antibodies inhibit the PEST domain of Mcl-1 from directly interacting with the pleckstrin homology (PH) domain of Akt; thus, such antibodies are useful as therapeutic agents for treating cancer.

In certain embodiments, the inhibitor is an antibody that specifically binds the PEST domain of Mcl-1 and prevents the PEST domain of Mcl-1 from directly interacting with the pleckstrin homology (PH) domain of Akt.

In certain embodiments, the inhibitor is an antibody that specifically binds pleckstrin homology (PH) domain of Akt and prevents the PEST domain of Mcl-1 from directly interacting with the pleckstrin homology (PH) domain of Akt. In certain contexts, an “antibody” refers to a protein based molecule that is naturally produced by animals in response to the presence of a protein or other molecule or that is not recognized by the animal's immune system to be a “self” molecule, i.e. recognized by the animal to be a foreign molecule and an antigen to the antibody. The immune system of the animal will create an antibody to specifically bind the antigen, and thereby targeting the antigen for elimination or degradation. It is well recognized by skilled artisans that the molecular structure of a natural antibody can be synthesized and altered by laboratory techniques. Recombinant engineering can be used to generate fully synthetic antibodies or fragments thereof providing control over variations of the amino acid sequences of the antibody. Thus, as used herein the term “antibody” is intended to include natural antibodies, monoclonal antibody, or non-naturally produced synthetic antibodies, bispecific antibodies, and binding fragments, such as single chain binding fragments. These antibodies may have chemical modifications. The term “monoclonal antibodies” refers to a collection of antibodies encoded by the same nucleic acid molecule that are optionally produced by a single hybridoma (or clone thereof) or other cell line, or by a transgenic mammal such that each monoclonal antibody will typically recognize the same antigen. The term “monoclonal” is not limited to any particular method for making the antibody, nor is the term limited to antibodies produced in a particular species, e.g., mouse, rat, etc.

From a structural standpoint, an antibody is a combination of proteins: two heavy chain proteins and two light chain proteins. The heavy chains are longer than the light chains. The two heavy chains typically have the same amino acid sequence. Similarly, the two light chains have the same amino acid sequence. Each of the heavy and light chains contain a variable segment that contains amino acid sequences which participate in binding to the antigen. The variable segments of the heavy chain do not have the same amino acid sequences as the light chains. The variable segments are often referred to as the antigen binding domains. The antigen and the variable regions of the antibody may physically interact with each other at specific smaller segments of an antigen often referred to as the “epitope.” Epitopes usually consist of surface groupings of molecules, for example, amino acids or carbohydrates. The terms “variable region,” “antigen binding domain,” and “antigen binding region” refer to that portion of the antibody molecule which contains the amino acid residues that interact with an antigen and confer on the antibody its specificity and affinity for the antigen. Small binding regions within the antigen-binding domain that typically interact with the epitope are also commonly alternatively referred to as the “complementarity-determining regions, or CDRs.”

Monoclonal antibodies directed against the antigen can be obtained from the immunized, transgenic mice using conventional hybridoma technology (see, e.g., U.S. Pat. No. 5,916,771). The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93, which is incorporated herein by reference in its entirety). For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety.

Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 6,311,415, 5,807,715, 4,816,567, and 4,816,397. Chimeric antibodies comprising one or more CDRs from a non-human species and framework regions from a human immunoglobulin molecule can be produced using a variety of techniques known in the art including, for example, CDR-grafting (EP 239,400; International Publication No. WO 91/09967; and U.S. Pat. Nos. 5,225,539, 5,530,101, and 5,585,089), veneering or resurfacing (EP 592,106; EP 519,596; Padlan, 1991, Molecular Immunology 28(4/5):489-498; Studnicka et al., 1994, Protein Engineering 7:805; and Roguska et al., 1994, Proc. Natl. Acad. Sci. USA 91:969), and chain shuffling (U.S. Pat. No. 5,565,332).

Chimeric antibodies include those having a variable region derived from a non-human antibody and a human immunoglobulin constant region. The term is also intended to include antibodies having a variable region derived from one human antibody grafted to an immunoglobulin constant region of a predetermined sequences or the constant region from another human for which there are allotypic differences residing in the constant regions of any naturally occurring antibody having the variable regions, e.g., CDRs 1, 2, and 3 of the light and heavy chain. Human heavy chain genes exhibit structural polymorphism (allotypes) that are inherited as a haplotype. The serologically defined allotypes differ within and between population groups. See Jefferis et al. mAb, 1 (2009), pp. 332-338.

Smith et al. report a protocol for the production of antigen-specific human chimeric antibodies wherein antibody-secreting cells (ASCs) are isolated from whole blood collected after vaccination and sorted by flow cytometry into single cell plates. Nat Protoc. 2009;4(3):372-84. The antibody genes of the ASCs are then amplified by RT-PCR and nested PCR, cloned into expression vectors and transfected into a human cell line. Meijer et al. report methods for isolation of human antibody repertoires with preservation of the natural heavy and light chain pairing. J Mol Biol. 2006 May 5;358(3):764-72. Wrammert et al. report using immunoglobulin variable regions isolated from sorted single ASCs to produce human monoclonal antibodies (mAbs) that bound with high affinity. Nature. 2008 May 29; 453(7195): 667-671.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized using conventional methodologies with a selected antigen.

### Methods of Use

In certain embodiments, this disclosure relates to methods of treating cancer comprising administering an effective amount of an inhibitor of Mcl-1 and Akt binding to a subject in need thereof. In certain embodiments, the inhibitor that prevents the PEST domain of Mcl-1 from directly interacting with the pleckstrin homology (PH) domain of Akt.

In certain embodiments, the subject is diagnosed with a cancer selected from lung, pancreatic, colorectal, uterine, esophageal, gastric, cervical, breast, prostate, or bladder cancer. In certain embodiments, the inhibitor of Mcl-1 and Akt binding is (PH-687) 2-(hydroxymethyl)-6-imino-2,3,3a,9a-tetrahydro-6H-furo[2′,3′:4,5] oxazolo[3,2-a]pyrimidin-3-yl dihydrogen phosphate, derivative, prodrug, or salt thereof.

In certain embodiments, the inhibitor of Mcl-1 and Akt binding is administered in combination with an additional chemotherapy agent. In certain embodiments, the inhibitor of Mcl-1 and Akt binding is (PH-687) 2-(hydroxymethyl)-6-imino-2,3,3a,9a-tetrahydro-6H-furo[2′,3′:4,5]oxazolo[3,2-a]pyrimidin-3-yl dihydrogen phosphate, derivative, prodrug, or salt thereof.

In certain embodiments, the subject is diagnosed with non-small cell lung cancer (NSCLC). In certain embodiments, malignant cells are seen on sputum cytology.

In certain embodiments, a tumor can be found with bronchoscopy or imaging tests.

In certain embodiments, a therapy disclosed herein may be instituted in addition to surgery to remove a portion of the lung such as a lobectomy, sleeve resection, segmentectomy, or wedge resection.

In certain embodiments, a therapy disclosed herein may be instituted in addition to radiation therapy.

In certain embodiments, a therapy disclosed herein may be instituted in a subject diagnosed with a gene mutation, such as a mutation in Akt, Mcl-1, EGFR, ALK, ROS1, BRAF, RET, MET, NTRK genes or combinations thereof.

In certain embodiments, the subject is diagnosed with an Akt at the PH-KD contact sites gene mutation (e.g., L52R, Q79K, and D323H). In certain embodiments, the subject is administered an inhibitor disclosed herein. In certain embodiments, the inhibitor is (PH-687) 2-(hydroxymethyl)-6-imino-2,3,3a,9a-tetrahydro-6H-furo[2′,3′:4,5]oxazolo[3,2-a]pyrimidin-3-yl dihydrogen phosphate, derivative, prodrug, or salt thereof. In certain embodiments, the inhibitor is an antibody.

In certain embodiments, the subject is diagnosed with an Mcl-1 at the PH-KD contact sites gene mutation. In certain embodiments, the subject is administered an inhibitor disclosed herein. In certain embodiments, the inhibitor is (PH-687) 2-(hydroxymethyl)-6-imino-2,3,3a,9a-tetrahydro-6H-furo[2′,3′:4,5]oxazolo[3,2-a]pyrimidin-3-yl dihydrogen phosphate, derivative, prodrug, or salt thereof. In certain embodiments, the inhibitor is an antibody.

In certain embodiments, the subject is diagnosed with an ALK gene mutation.

In certain embodiments, the subject is administered an inhibitor disclosed herein in combination with another ALK inhibitor. In certain embodiments, the inhibitor is (PH-687) 2-(hydroxymethyl)-6-imino-2,3,3a,9a-tetrahydro-6H-furo[2′,3′:4,5]oxazolo[3,2-a]pyrimidin-3-yl dihydrogen phosphate, derivative, prodrug, or salt thereof. In certain embodiments, the inhibitor is an antibody.

In certain embodiments, the subject is administered an inhibitor disclosed herein in combination with crizotinib, alectinib, brigatinib, lorlatinib, foretinib, alvotinib, belizatinib, repotrectinib, entrectinib, or ensartinib. In certain embodiments, the inhibitor is (PH-687) 2-(hydroxymethyl)-6-imino-2,3,3a,9a-tetrahydro-6H-furo[2′,3′:4,5]oxazolo[3,2-a]pyrimidin-3-yl dihydrogen phosphate, derivative, prodrug, or salt thereof. In certain embodiments, the inhibitor is an antibody.

In certain embodiments, the subject is diagnosed with an EGFR gene mutation.

In certain embodiments, the subject is administered an inhibitor disclosed herein in combination with another EGFR inhibitor. In certain embodiments, the inhibitor is (PH-687) 2-(hydroxymethyl)-6-imino-2,3,3a,9a-tetrahydro-6H-furo[2′,3′:4,5]oxazolo[3,2-a]pyrimidin-3-yl dihydrogen phosphate, derivative, prodrug, or salt thereof. In certain embodiments, the inhibitor is an antibody.

In certain embodiments, the subject is administered an inhibitor disclosed herein in combination with afatinib, erlotinib, or lapatinib. In certain embodiments, the inhibitor is (PH-687) 2-(hydroxymethyl)-6-imino-2,3,3a,9a-tetrahydro-6H-furo[2′,3′:4,5]oxazolo[3,2-a]pyrimidin-3-yl dihydrogen phosphate, derivative, prodrug, or salt thereof. In certain embodiments, the inhibitor is an antibody.

In certain embodiments, the subject is diagnosed with a ROS1 gene mutation.

In certain embodiments, the subject is administered an inhibitor disclosed herein in combination with crizotinib, entrectinib, or ceritinib. In certain embodiments, the inhibitor is (PH-687) 2-(hydroxymethyl)-6-imino-2,3,3a,9a-tetrahydro-6H-furo[2′,3′:4,5]oxazolo[3,2-a] pyrimidin-3-yl dihydrogen phosphate, derivative, prodrug, or salt thereof. In certain embodiments, the inhibitor is an antibody.

In certain embodiments, the subject is diagnosed with a BRAF gene mutation.

In certain embodiments, the subject is administered an inhibitor disclosed herein in combination with dabrafenib or trametinib. In certain embodiments, the inhibitor is (PH-687) 2-(hydroxymethyl)-6-imino-2,3,3a,9a-tetrahydro-6H-furo[2′,3′:4,5]oxazolo[3,2-a]pyrimidin-3-yl dihydrogen phosphate, derivative, prodrug, or salt thereof. In certain embodiments, the inhibitor is an antibody.

In certain embodiments, the subject is diagnosed with a RET gene mutation. In certain embodiments, the subject is administered an inhibitor disclosed herein in combination with selpercatinib or pralsetinib. In certain embodiments, the inhibitor is (PH-687) 2-(hydroxymethyl)-6-imino-2,3,3a,9a-tetrahydro-6H-furo[2′,3′:4,5]oxazolo[3,2-a]pyrimidin-3-yl dihydrogen phosphate, derivative, prodrug, or salt thereof. In certain embodiments, the inhibitor is an antibody.

In certain embodiments, the subject is diagnosed with a MET gene mutation.

In certain embodiments, the subject is administered an inhibitor disclosed herein in combination with capmatinib. In certain embodiments, the inhibitor is (PH-687) 2-(hydroxymethyl)-6-imino-2,3,3a,9a-tetrahydro-6H-furo[2′,3′:4,5]oxazolo[3,2-a]pyrimidin-3-yl dihydrogen phosphate, derivative, prodrug, or salt thereof. In certain embodiments, the inhibitor is an antibody.

In certain embodiments, the subject is diagnosed with a NTRK gene mutation

In certain embodiments, the subject is administered an inhibitor disclosed herein in combination with larotrectinib or entrectinib. In certain embodiments, the inhibitor is (PH-687) 2-(hydroxymethyl)-6-imino-2,3,3a,9a-tetrahydro-6H-furo[2′,3′:4,5]oxazolo[3,2-a]pyrimidin-3-yl dihydrogen phosphate, derivative, prodrug, or salt thereof. In certain embodiments, the inhibitor is an antibody.

In certain embodiments, the subject is diagnosed with tumors or cancer cells with higher than normal levels of PD-L1.

In certain embodiments, the subject is administered an inhibitor disclosed herein in combination with pembrolizumab, atezolizumab, nivolumab, or ipilimumab. In certain embodiments, the inhibitor is (PH-687) 2-(hydroxymethyl)-6-imino-2,3,3a,9a-tetrahydro-6H-furo[2′,3′:4,5]oxazolo[3,2-a]pyrimidin-3-yl dihydrogen phosphate, derivative, prodrug, or salt thereof. In certain embodiments, the inhibitor is an antibody.

In certain embodiments, the subject is administered an inhibitor disclosed herein in combination with bevacizumab. In certain embodiments, the inhibitor is (PH-687) 2-(hydroxymethyl)-6-imino-2,3,3a,9a-tetrahydro-6H-furo[2′,3′:4,5]oxazolo[3,2-a]pyrimidin-3-yl dihydrogen phosphate, derivative, prodrug, or salt thereof. In certain embodiments, the inhibitor is an antibody.

In certain embodiments, the subject is diagnosed with squamous cell NSCLC.

In certain embodiments, the subject is administered an inhibitor disclosed herein in combination with necitumumab. In certain embodiments, the inhibitor is (PH-687) 2-(hydroxymethyl)-6-imino-2,3,3a,9a-tetrahydro-6H-furo[2′,3′:4,5]oxazolo[3,2-a]pyrimidin-3-yl dihydrogen phosphate, derivative, prodrug, or salt thereof. In certain embodiments, the inhibitor is an antibody.

### Pharmaceutical Compositions

In certain embodiments, this disclosure relates to pharmaceutical compositions comprising inhibitors disclose herein and a pharmaceutically acceptable excipient. In certain embodiments, the pharmaceutically acceptable excipient is selected from a diluent, disintegrant, solubilizing agent, or a lubricant.

In certain embodiments, the pharmaceutically acceptable excipient is selected from a saccharide, disaccharide, sucrose, lactose, glucose, mannitol, sorbitol, polysaccharides, starch, cellulose, microcrystalline cellulose, cellulose ether, hydroxypropyl cellulose (HPC), xylitol, maltitol, gelatin, polyvinylpyrrolidone (PVP), polyethylene glycol (PEG), hydroxypropyl methylcellulose (HPMC), crosslinked sodium carboxymethyl cellulose, dibasic calcium phosphate, calcium carbonate, stearic acid, magnesium stearate, talc, magnesium carbonate, silica, vitamin A, vitamin E, vitamin C, retinyl palmitate, selenium, cysteine, methionine, citric acid, and sodium citrate, methyl paraben, propyl paraben, and combinations thereof.

In certain embodiments, the pharmaceutically acceptable excipient is a diluent. Examples include microcrystalline cellulose, other diluents may be, for example: calcium carbonate, calcium phosphate, calcium sulfate, cellulose acetate, erythritol, ethylcellulose, fructose, inulin, isomalt, lactitol, lactose, magnesium carbonate, magnesium oxide, maltitol, maltodextrin, maltose, mannitol, polydextrose, polyethylene glycol, pullulan, simethicone, sodium bicarbonate, sodium carbonate, sodium chloride, sorbitol, starch, sucrose, trehalose and xylitol.

In certain embodiments, the pharmaceutically acceptable excipient is a disintegrant. Examples of a disintegrant may be, for example: alginic acid, calcium alginate, carboxymethylcellulose calcium, chitosan, colloidal silicon dioxide, croscarmellose sodium, crospovidone, glycine, guar gum, hydroxypropyl cellulose, low-substituted hydroxypropyl cellulose, magnesium aluminum silicate, methylcellulose, povidone, sodium alginate, sodium carboxymethylcellulose, sodium starch glycolate and starch.

In certain embodiments, the pharmaceutically acceptable excipient is a solubilizing agent. Examples of a solubilizing agent may be, for example: benzalkonium chloride, benzyl benzoate, sulfobutyl ether β-cyclodextrin sodium, cetylpyridinium chloride, cyclodextrins, diethylene glycol monoethyl ether, fumaric acid, hydroxypropyl beta cyclodextrin, hypromellose, lanolin alcohols, lecithin, oleyl alcohol, phospholipids, poloxamer, polyoxyethylene alkyl ethers, polyoxyethylene castor oil derivatives, polyoxyethylene sorbitan fatty acid esters, polyoxyl hydroxystearate, polyoxylglycerides, povidone, pyrrolidone, sodium lauryl sulfate, sorbitan esters (sorbitan fatty acid esters), tricaprylin, triolein and vitamin E polyethylene glycol succinate.

In certain embodiments, the pharmaceutically acceptable excipient is a lubricant. Examples of a lubricant may be, for example calcium stearate, glyceryl behenate, glyceryl dibehenate, glyceryl monostearate, glyceryl palmitostearate, a mixture of behenate esters of glycerine (e.g. a mixture of glyceryl dibehenate, tribehenin and glyceryl behenate), leucine, magnesium stearate, myristic acid, palmitic acid, poloxamer, polyethylene glycol, potassium benzoate, sodium benzoate, sodium lauryl sulfate, sodium stearate, sodium stearyl fumarate, stearic acid, talc, tribehenin and zinc stearate.

In certain embodiments, the pharmaceutically acceptable excipient is selected from lactose, sucrose, mannitol, triethyl citrate, dextrose, cellulose, methyl cellulose, ethyl cellulose, hydroxyl propyl cellulose, hydroxypropyl methylcellulose, carboxymethylcellulose, croscarmellose sodium, polyvinyl N-pyrrolidone, crospovidone, ethyl cellulose, povidone, methyl and ethyl acrylate copolymer, polyethylene glycol, fatty acid esters of sorbitol, lauryl sulfate, gelatin, glycerin, glyceryl monooleate, silicon dioxide, titanium dioxide, talc, corn starch, carnauba wax, stearic acid, sorbic acid, magnesium stearate, calcium stearate, castor oil, mineral oil, calcium phosphate, starch, carboxymethyl ether of starch, iron oxide, triacetin, acacia gum, esters, or salts thereof.

In certain embodiments, the pharmaceutical composition is in the form of a tablet, pill, capsule, gel, gel capsule or cream. In certain embodiments, the pharmaceutical composition is in the form of a sterilized pH buffered aqueous salt solution or a saline phosphate buffer between a pH of 6 to 8, optionally comprising a saccharide or polysaccharide.

In certain embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art.

For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange.

Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

Pharmaceutically acceptable salts derived from appropriate bases include alkali metal, alkaline earth metal, ammonium, or quaternary ammonium, e.g., N⁺(C₁₋₄alkyl)₄, salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate. Organic bases from which salts can be derived include, for example, primary, secondary, and tertiary amines, substituted amines including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

In certain embodiments, an inhibitor disclosed herein may be used in the “free base form” or as a pharmaceutically acceptable salt, or as any mixture thereof. In one embodiment the inhibitor is in the free base form. It is understood that “free base form” refers to the case where the inhibitor is not in the form of a salt.

In certain embodiments, this disclosure relates to the production of a medicament comprising an inhibitor for use in treating cancer.

In certain embodiments, this disclosure relates to kits or pharmaceutical packaging comprising combinations of agents disclosed herein with instructions for use. In certain embodiments, the individual agent may be packaged in a container, e.g., vial, box, syringe, or bottle. In certain embodiments, instructions may be in a pamphlet inside a container or on the outside or inside of the container.

### Mcl-1 Interacts with Akt to Promote Lung Cancer Progression

It has been discovered that Mcl-1 directly interacts via its PEST domain with Akt at the PH domain, which disrupts intramolecular interactions between the PH domain and KD of Akt, leading to phosphorylation and activation of Akt and acceleration of lung cancer cell growth in vitro and in vivo. A small-molecule PH-687 has been identified that specifically targets the PH domain of Akt (i.e., the Mcl-1-binding region), disrupts Mcl-1/Akt interaction, inhibits Akt activity, and exhibits strong antitumor activity against NSCLC in vitro and in vivo.

The PI3K/Akt pathway regulates multiple cellular functions, including cell growth, differentiation, proliferation, survival, motility, invasion, and intracellular trafficking. Akt is frequently activated in cancers, which occurs through mutations of upstream genes like PIK3CA, PTEN, and KRAS. Akt can also be activated through mutations within its PH domain. Mcl-1 activates Akt in a mechanism independent of genomic alterations. Depletion of endogenous Mcl-1 from human lung cancer cells using CRISPR/Cas9 or Mcl-1 shRNA resulted in significant downregulation of Akt activity and suppression of lung cancer cell growth in vitro and in vivo. In contrast, overexpression of exogenous Mcl-1 enhanced Akt activity and promoted lung cancer cell growth. Analysis of 208 patients with NSCLC tumors revealed that higher levels of Mcl-1 are correlated with higher levels of pAkt in tumor tissues, which are associated with poor outcome of patients with NSCLC. These findings strongly suggest that Mcl-1, in addition to its canonical antiapoptotic function via interaction with proapoptotic Bcl2 family proteins, may also directly regulate Akt to support cancer cell growth, which may negatively affect the prognosis of patients with NSCLC.

Mechanistically, Mcl-1 directly interacts with Akt in lung cancer cells and in a cell-free system. Interaction between the PH and KD is important for maintaining the kinase in an inactive state. Mutations observed in human tumors in Akt at the PH-KD contact sites (i.e., L52R, Q79K, and D323H) can disrupt PH-KD interaction, leading to activation of Akt in human cancers. Domain-mapping studies reveal that Mcl-1 interacts directly via its PEST domain with Akt at the PH domain, and the binding of Mcl-1 with the PH domain can disrupt PH/KD interaction, leading to Akt activation. Importantly, Mcl-1/PH interaction not only activates Akt, but also promotes lung cancer cell growth in vitro and in vivo. These findings indicate a mechanism of Akt activation that occurs through a PH mutation(s)-independent manner.

Mcl-1 interacts directly via its PEST domain with Akt at the PH domain, leading to disruption of intramolecular PH/KD interactions, which results in Akt activation in cancer cells. Thus, Mcl-1, in addition to its antiapoptotic function, appears to function upstream of Akt and activates Akt signaling via direct interaction. Specifically targeting Mcl-1/Akt interaction by employing antibodies or small molecules and such as PH-687 represents an effective strategy for cancer treatment.

### Mcl-1 Loss Leads to Growth Inhibition of Cancer Cells, Which May Occur Through Downregulation of Akt Activity

To test the effects of Mcl-1 on cancer cell growth, endogenous Mcl-1 was knocked out from human lung cancer H1299 cells using CRISPR/Cas9 technology. Cell growth and colony formation were compared in H1299 parental versus H1299 Mcl-1⁻/⁻ cells. Results indicate that depletion of endogenous Mcl-1 resulted in significant growth inhibition of H1299 cells.

Protein kinase-mediated signaling pathways play roles in regulating cancer cell growth. To assess whether Mcl-1 regulates protein kinase-mediated signaling pathways in human lung cancer cells, a human phosphokinase array was employed to simultaneously detect the relative levels of phosphorylation of 43 kinase phosphorylation sites using total proteins isolated from H1299 parental versus H1299 Mcl-1⁻/⁻ cells. Intriguingly, among 43 kinases, significantly decreased levels of Akt phosphorylation at S473 and T308 were observed in H1299 Mcl-1⁻/⁻ cells as compared with H1299 parental cells. No significant changes in phosphorylation levels of other kinases were observed in H1299 parental versus H1299 Mcl-1⁻/⁻ cells. These results suggest that Akt may act as a downstream signaling kinase in Mcl-1-mediated enhancement of cancer cell growth.

Akt activates mTOR/S6K signaling to promote cell growth. Experiments were performed to determine whether Mcl-1 loss affects the Akt downstream signaling pathway. Knockout of Mcl-1 from H1299 cells or knockdown of Mcl-1 from another lung cancer cell line H460 cells resulted in decreased levels of phosphorylation: pAkt, pmTOR, and pS6K. These results suggest that Mcl-1 positively regulates Akt/mTOR signaling.

The canonical antiapoptotic function of Mcl-1 occurs through interaction with proapoptotic proteins of Bcl2 family proteins. Activation of Akt may be a mechanism by which Mcl-1 regulates apoptosis and proliferation. In addition to cell growth, cell proliferation, and caspase-3/7 activity were also measured in H1299 parental and H1299 Mcl-1⁻/⁻ cells. Results indicate that knockout of Mcl-1 resulted in decreased cell proliferation and increased caspase-3/7 activity.

To demonstrate whether Mcl-1 regulation of cancer cell growth occurs through Akt, a constitutively active form of Akt (Myr HAAkt1) or empty vector was transfected into H1299 Mcl-1⁻/⁻ cells, followed by colony formation assay. Knockout of Mcl-1 resulted in growth inhibition and expression of the constitutively active form of Akt in H1299 Mcl-1⁻/⁻ cells restored cell growth.

### Mcl-1 Loss Retards Tumor Growth In Vivo

To evaluate the role of Mcl-1 in tumor growth in vivo, the same number (3×10⁶) of H1299 parental or Mcl-1⁻/⁻ H1299 cells were injected into subcutaneous tissue in the flank region of nude mice to generate lung cancer xenografts. Tumor volume was measured once every 3 days. Results indicate that depletion of Mcl-1 significantly inhibited growth of xenografted tumors (FIG. 1), suggesting that Mcl-1 is an important molecule for tumor growth.

IHC staining revealed that Mcl-1 deficiency was associated with decreased levels of pAkt and Ki67 (i.e., cell proliferation marker) and increased active caspase-3 (i.e., apoptosis marker) in tumor tissues. Decreased pAkt levels were also observed in H460 xenograft tumors expressing control shRNA or human Mcl-1 shRNA. These results indicate that Mcl-1/Akt signaling plays an important role in tumor growth.

### The PEST Domain of Mcl-1 Interacts with the PH Domain of Akt

To evaluate the mechanism by which Mcl-1 regulates Akt, coimmunoprecipitation (co-IP) experiments using Mcl-1 or Akt antibody were performed in H1299 parental versus H1299 Mcl-1⁻/⁻ cells. Results indicate that Mcl-1 was associated with Akt protein in H1299 parental cells. Because Akt antibody could pull-down Mcl-1 protein only in H1299 parental, but not in H1299 Mcl-1⁻/⁻ cells, this indicates a potential direct interaction between Mcl-1 and Akt in human lung cancer cells. To further verify this interaction, similar co-IP experiments were carried out in a cell-free system using purified recombinant Mcl-1 and Akt proteins. Consistently, Mcl-1 also directly interacted with Akt in the cell-free system. To detect the intracellular localization(s) of Akt/Mcl-1 interaction, a Duolink™ proximity ligation assay (PLA) system was employed to detect Akt/Mcl-1 interaction in H1299 cells and H460 cells according to the manufacturer's instructions. Two primary antibodies raised in different species (i.e., Mcl-1 antibody from rabbit and Akt antibody from mouse) are used to detect two unique protein targets. A pair of oligonucleotide-labelled secondary antibodies (PLA probes) then binds to the primary antibodies. Hybridizing connector oligos join the PLA probes only if they are in close proximity to each other and ligase forms a closed circle DNA template that is required for rolling-circle amplification. The PLA probe then acts as a primer for a DNA polymerase, which generates concatemeric sequences during rolling-circle amplification. This allows up to 1,000-fold amplified signal that is still tethered to the PLA probe, allowing localization of the signal. Labelled oligos hybridize to the complementary sequences within the amplicon. PLA signals were detected by fluorescence microscopy as discrete spots. Intriguingly, clear discrete spots were observed mainly in the cytoplasm, indicating that Mcl-1/Akt interactions in H1299 and H460 cells occur mainly in the cytoplasm. IgG, single Akt antibody alone, and single Mcl-1 antibody alone were used as negative controls and no discrete spots were observed.

Mcl-1 contains multiple functional domains, including N-terminal, PEST, BH1, BH2, BH3, and transmembrane (TM) domains. To identify the binding region of Mcl-1 to Akt, a panel of Flag-tagged Mcl-1 deletion mutants, including DN (10-120), DPEST (120-200), DBH1 (256-265), DBH2 (305-315), DBH3 (213-221), and DTM (329-346), were employed for co-transfections with HA-Akt and co-IP experiments using a Flag antibody. Results revealed that WT, DN, DBH1, DBH2, DBH3, and DTM but not DPEST Mcl-1 mutants, directly interacted with Akt protein, indicating that the PEST domain comprises the Akt-binding site on Mcl-1 protein. Conversely, to further identify the Mcl-1 binding site on Akt protein, a series of GST-tagged Akt deletion mutants, including 1 (GST-PH domain), 2, 3, 4, 5, 6 (GST-kinase domain), 7, and 8 (WT), were used in co-transfections with Flag-tagged WT Mcl-1 and GST pull-down experiments. Intriguingly, WT (8) and Akt deletion mutants containing the PH domain (1, 3, and 7), but not Akt deletion mutants lacking the PH domain (2, 4, 5, and 6), could interact with WT Mcl-1. These results demonstrate that the PH domain of the Akt protein is the Mcl-1 binding region.

### Mcl-1/Akt Binding is Essential for Mcl-1 Disruption of Intramolecular PH/KD Interactions, Activation of Akt, and Promotion of Tumor Growth

Intramolecular PH domain/KD interactions are important in maintaining AKT in an inactive state, and AKT activation is trigged by a conformational change that dislodges the PH from the KD. Because Mcl-1 can directly interact with Akt at the PH domain, experiments were designed to test whether Mcl-1 affects the intramolecular PH domain/KD interaction. GST-tagged KD, GFP-tagged PH domain, and Flag-tagged WT Mcl-1 or DPEST Mcl-1 mutant were used for co-transfection and co-IP experiments. Results reveal that exogenous expression of WT Mcl-1 but not the PEST deletion mutant (DPEST) significantly reduced the interaction between PH domain and KD. To further confirm these findings, a mammalian two-hybrid system was employed to measure the PH/KD interaction. Co-transfection of Gal4BD-Akt KD and VP16AD-Akt PH along with pG5SEAP in cells drives the expression of secreted alkaline phosphatase (SEAP) when KD/PH interaction occurs. To test the effect of Mcl-1 on KD/PH interaction, the two-hybrid H1299 Mcl-1⁻/⁻ cells were co-transfected with WT Mcl-1 or DPEST mutant Mcl-1, followed by analysis of SEAP activity. WT Mcl-1, but not DPEST mutant, significantly reduced SEAP activity which further confirmed the co-IP results. Because the PEST domain is required for Mcl-1/Akt binding, it is proposed that Mcl-1 disruption of intramolecular PH domain/kinase domain interactions may occur through Mcl-1/Akt binding.

PIP3 directly binds to the PH domain and induces a conformational change in Akt, which enables PDK1 or mTORC2 to access and phosphorylate Akt at T308 or S473. To test whether Mcl-1, similar to PIP3, may also affect the interactions between Akt and PDK1 or mTORC2, co-IP experiments using Akt antibody were performed in H1299 parental cells or H1299 Mcl-1⁻/⁻ cells expressing WT Mcl-1, DPEST Mcl-1 mutant, or empty vector, followed by Western blot using PDK1 or mTORC2 antibody, respectively. Results reveal that knockout of Mcl-1 disrupted Akt/PDK1 and Akt/mTORC2 interactions, and expression of WT Mcl-1, but not PEST deletion mutant, restored Akt/PDK1 and Akt/mTORC2 interactions.

To test whether Mcl-1/Akt binding is needed for Mcl-1 regulation of Akt activity and cancer cell growth, WT Mcl-1 and PEST domain deletion mutant Mcl-1 (DPEST) were exogenously expressed in H1299 Mcl-1⁻/⁻ cells, followed by analysis of pAkt, pmTOR, p-p70 S6K, and colony formation. Expression of exogenous WT Mcl-1 but not the DPEST mutant Mcl-1 led to increased levels of pAkt, pmTOR, and p-p70 S6K and promotion of cancer growth. To further assess whether deletion of PEST domain (i.e., Akt binding site) affects tumor growth, Mcl-1-deficient H1299 cells expressing exogenous WT or DPEST mutant were employed to establish lung cancer xenografts. Expression of WT Mcl-1 significantly promoted tumor growth in xenograft models. Deletion of PEST from Mcl-1 ablated the capacity of Mcl-1 to promote tumor growth (FIG. 2). These results indicate that Mcl-1/Akt binding is required for Mcl-1 to activate Akt and promote cancer growth.

### Mcl-1 Expression Levels are Correlated with Phosphorylated Akt (pAkt) Levels in Tumor Tissues from Patients with NSCLC

The majority of experiments employed human lung cancer cell lines (i.e., H1299 or H460) derived from non-small cell lung cancer (NSCLC); therefore, it was of interest to test whether Mcl-1 and pAkt are upregulated in tumor tissues from patients with NSCLC, and whether Mcl-1 expression is correlated with pAkt in NSCLC patient tumors. Mcl-1 and pAkt were analyzed in samples from 208 patients with NSCLC by IHC staining employing anti-Mcl-1 or phospho-specific Akt (S473) antibody, respectively. Tissue microarrays were generated with replicate cores of tumor and adjacent normal lung. Semiquantitative evaluation of IHC staining of Mcl-1 or pAkt was carried out using immunoscores based on both percentage of stained cells and staining intensity. Mcl-1 protein expression was significantly higher in tumor tissues compared with adjacent normal lung tissues. The observed levels of Mcl-1 were positively correlated with levels of pAkt in tumor tissues. Importantly, elevated levels of Mcl-1 or pAkt in tumor tissues were significantly associated with poor overall survival for patients with NSCLC, suggesting that Mcl-1 and pAkt, could be potential prognostic biomarkers of survival for patients with NSCLC.

### Small-Molecule PH-687 Targets the PH Domain, Disrupts Mcl-1/Akt Interaction, and Inhibits Akt Activity Leading to Growth Inhibition of Human Lung Cancer Cells

Interaction of Mcl-1 via its PEST domain with the PH domain of Akt leads to Akt activation by disrupting intramolecular interactions between its PH domain and KD, indicating that the PH domain is an attractive target for screening of small molecules that may potentially disrupt Mcl-1/Akt binding. An NCI database library of 300,000 small molecules was docked into the PH structure pocket (aa 6-108) identified by the UCSF DOCK 6.1 program suite for screening.

The small molecules were ranked according to their energy scores. The top 500 small molecules based on predicted binding energies were selected for screening of cytotoxicity in human lung cancer cells by sulforhodamine B assay as described in Park et al. Novel small molecule inhibitors of Bcl-XL to treat lung cancer. Cancer Res 2013;73: 5485-96. Among these small molecules, the compound (PH-687) 2-(hydroxymethyl)-6-imino-2,3,3a,9a-tetrahydro-6H-furo[2′,3′:4,5]oxazolo [3,2-a]pyrimidin-3-yl dihydrogen phosphate had the most potent activity against human lung cancer cells.

To test whether PH-687 specifically binds to the PH domain in Akt, recombinant GST-tagged WT Akt, PH deletion Akt mutant (DPH), and PH domain-only proteins were generated and PH-687 compound/Akt protein binding were measured using a thermal shift assay. Thermal shift assay is a technique to study protein/small-molecule interactions that enhance protein thermal stability and increase melting temperatures as described in Jin et al. Glutamate dehydrogenase 1 signals through antioxidant glutathione peroxidase 1 to regulate redox homeostasis and tumor growth. Cancer Cell, 2015, 27:257-70. Dose-dependent increases in Tm were observed when purified GST-WT Akt and GST-PH-mutant proteins were incubated with increasing concentrations of PH-678. There was no significant increase in Tm when GST-DPH protein was incubated with increasing concentrations of PH-687. These results indicate that small molecule PH-687 binds to the PH domain of Akt protein, and the PH domain is essential for this binding. Because PH-687 can bind to the Mcl-1-binding site (i.e., PH domain) on Akt protein, the effect of PH-687 on Mcl-1/Akt interaction was tested. Co-IP experiments reveal that treatment of H1299 cells with increasing concentrations of PH-687 resulted in a dose-dependent dissociation of Mcl-1/Akt complexes (FIG. 3A), indicating that PH-687 is able to disrupt Mcl-1/Akt interaction in H1299 cells. Importantly, PH-678-induced disruption of Mcl-1/Akt interaction significantly reduced Akt activity leading to growth inhibition in various human lung cancer cell lines.

To test whether PH-687-mediated Mcl-1/Akt dissociation enhances interaction between the PH and KD domains, a mammalian two-hybrid system was employed to measure the PH/KD interaction in H1299 cells using Gal4BD-Akt KD, VP16AD-Akt PH, and pG5SEAP in the absence or presence of PH-687. Results reveal that treatment of H1299 cells with increasing concentrations (0, 0.5, 1, 2 mmol/L) of PH-687 for 24 hours led to increased PH/KD interaction in a dose-dependent manner. Intriguingly, treatment of H1299 cells with PH-687 also resulted in a dose dependent decreased interaction of Akt with PDK1 or mTORC2.

To further test whether PH-687 specifically functions through targeting Mcl-1/AKT signaling in lung cancer cells, H1299 parental cells, H1299 Mcl-1⁻/⁻ cells and H1299 cells expressing Akt siRNA were treated with increasing concentrations of PH-687 (0, 0.1, 0.5, 1 mmol/L), followed by colony formation assay. Results reveal that treatment of H1299 cells with increasing concentrations of PH-687 led to growth inhibition in a dose-dependent manner. In contrast, knockout of Mcl-1 by CRISPR/cas9 or knockdown of Akt by Akt siRNA reduced the inhibitory effect of PH-687 on cell growth indicating that the effect of PH-687 occurs through Mcl-1/Akt signaling.

To test whether Mcl-1 or PH-687 also affects Akt activation by growth factors such as EGF, IGF, or insulin, first, H1299 parental and H1299 Mcl-1⁻/⁻ cells were treated with EGF (100 ng/mL), IGF (10 ng/mL), or insulin (1 mg/mL) for 1 hour, followed by analysis of Akt phosphorylation. Results indicate that treatment of H1299 cells with EGF, IGF, and insulin enhanced levels of pAkt S473 and pAkt T308. Knockout of Mcl-1 blocked EGF, IGF, and insulin-induced Akt phosphorylation at S473 and T308. Second, H1299 cells were treated with EGF (100 ng/mL), IGF (10 ng/mL), or insulin (1 mg/mL) in the absence or presence of PH-687 (2 mmol/L) for 1 hour, followed by analysis of Akt phosphorylation. Results reveal that PH-687 inhibited EGF, IGF, and insulin-induced Akt phosphorylation at S473 and T308.

### PH-687 has Potent Antitumor Activity Against NSCLC In Vivo

To test the antitumor potency of PH-687 in vivo, nude mice with NSCLC (i.e., H1299) xenografts were treated with increasing doses (0, 30, 60, and 90 mg/kg/day) of PH-687 for 4 weeks. Treatment with PH-687 suppressed tumor growth in a dose-dependent manner in vivo (FIG. 4). IHC staining of representative samples from harvested tumor tissues revealed that treatment of mice with PH-687 resulted in decreased levels of pAkt (S473), pAkt (T308), and Ki67 in association with increased apoptosis (i.e., active caspase-3). There was no significant weight loss and no significant increase in alanine aminotransferase, aspartate aminotransferase, and blood urea nitrogen or reduction in WBC, RBC, hemoglobin, and PLT in mice treated with PH-687 in the dose range of 30-90 mg/kg/day.

### Treatment of Lung Cancer Xenografts

Six-week-old male nude mice were purchased from Harlan and housed under pathogen-free conditions. H1299 cells were subcutaneously implanted into mouse flanks. Tumor bearing mice were randomly grouped and tumors were allowed to grow to an average volume of 100 mm3 before treatment. Mice were treated with PH-687 through intraperitoneal injection (i.p) at the indicated dose. During treatment, tumor volumes were measured by caliper once every 4 days.

### Human Patient Samples and IHC Staining

Paraffin-embedded human lung tissue samples from 208 patients with NSCLC were obtained. Tissue microarray was constructed with replicate cores of tumor and adjacent normal lung. After deparaffinization, rehydration, inactivation of endogenous peroxidase, and antigen retrieval, IHC staining was performed. The following primary antibody dilutions were used: anti-Mcl-1 (1:300), anti-pAKT S473 (1:100), anti-pAKT T308 (1:100), anti-Ki67 (1:500), and anti-active caspase-3 (1:100). Ki67-positive cells and active caspase-3-positive cells in tumor tissues were scored. Percentage of positive cells was determined from three separate fields in each of three independent tumor samples. The semiquantitative evaluation of IHC staining of Mcl-1 and pAkt was carried out using an immunoscore based on both the percentage of stained cells and staining intensity.

1. A method of treating cancer comprising administering an effective amount of an inhibitor of Mcl-1 and Akt binding to a human subject in need thereof.

2. The method of claim 1 wherein the inhibitor of Mcl-1 and Akt binding is an inhibitor that prevents the PEST domain of Mcl-1 from directly interacting with the pleckstrin homology (PH) domain of Akt.

3. The method of claim 1 wherein the inhibitor is 2-(hydroxymethyl)-6-imino-2,3,3a,9a-tetrahydro-6H-furo[2′,3′:4,5]oxazolo[3,2-a]pyrimidin-3-yl dihydrogen phosphate, derivative, ester, or salt thereof.

4. The method of claim 1 wherein the inhibitor is a compound of formula I, [chemical structure]
derivative, ester, or salt thereof, wherein:
Q is O or S;
U is a linking group, O, S, NH, or CH₂;
X is O, S, NH, or CH₂;
Y is O, S NH, or CH₂;
Z is N or CH;
R¹, R², R³, R⁴, R⁵, R⁶, R⁷, and R⁸, are each individually and independently hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, phosphate, alkylsulfinyl, alkylsulfonyl, arylsulfonyl, carbocyclyl, aryl, or heterocyclyl, wherein R¹, R², R³, R⁴, R⁵, R⁶, R⁷, and R⁸ are optionally substituted with one or more, the same or different, R¹⁰;
R¹⁰ is alkyl, halogen, cyano, hydroxy, amino, mercapto, formyl, carboxy, carbamoyl, alkoxy, alkanoyl, alkylthio, alkylamino, phosphate, aminoalkyl, (alkyl)₂amino, alkylsulfinyl, alkylsulfonyl, arylsulfonyl, carbocyclyl, aryl, or heterocyclyl, wherein R¹⁰ is optionally substituted with one or more, the same or different, R¹¹;
R¹¹ is alkyl, halogen, cyano, hydroxy, amino, mercapto, formyl, carboxy, carbamoyl, alkoxy, alkanoyl, alkylthio, alkylamino, aminoalkyl, (alkyl)₂amino, phosphate, alkylsulfinyl, alkylsulfonyl, arylsulfonyl, carbocyclyl, aryl, or heterocyclyl, wherein R¹¹ is optionally substituted with one or more, the same or different, R¹²; and
R¹² is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, 2-methoxyethoxy, 2-hydroxyethoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

5. The method of claim 4 wherein R¹ is alkyl substituted with hydroxy.

6. The method of claim 4 wherein R³ is hydroxy.

7. The method of claim 4 wherein R⁴ is hydroxy.

8. The method of claim 1 wherein the subject is diagnosed with non-small cell lung cancer.

9. The method of claim 1, wherein the inhibitor of Mcl-1 and Akt binding is administered in combination with an additional chemotherapy agent.

10. A pharmaceutical composition comprising an inhibitor of Mcl-1 and Akt binding disclosed herein or pharmaceutically acceptable salt thereof and a pharmaceutically acceptable excipient.

11. The pharmaceutical composition of claim 10 in the form of a pill, capsule, or table.

12. The pharmaceutical composition of claim 10 in the form of an aqueous isotonic or non-isotonic pH buffered solution.

13. The pharmaceutical composition of claim 10, wherein the pharmaceutically acceptable excipient is selected from a saccharide, disaccharide, sucrose, lactose, glucose, mannitol, sorbitol, polysaccharides, starch, cellulose, microcrystalline cellulose, cellulose ether, hydroxypropyl cellulose (HPC), xylitol, maltitol, gelatin, polyvinylpyrrolidone (PVP), polyethylene glycol (PEG), hydroxypropyl methylcellulose (HPMC), crosslinked sodium carboxymethyl cellulose, dibasic calcium phosphate, calcium carbonate, stearic acid, magnesium stearate, talc, magnesium carbonate, silica, vitamin A, vitamin E, vitamin C, retinyl palmitate, selenium, cysteine, methionine, citric acid, and sodium citrate, methyl paraben, propyl paraben, and combinations thereof.
